(19) United States
(12) Patent Application Publication
(10) Pub. No.: US 2023/0087383 A1
(43) Pub. Date: Mar. 23, 2023

(54) Platelet Activation Using Specific Frequencies

(71) Applicant: CELL CODE, LLC, Mill Valley, CA (US)

(72) Inventors: Wayne J. Powell, Centennial, CO (US); Beth McDougall, Mill Valley, CA (US)

(21) Appl. No.: 17/802,079

(22) PCT Filed: Feb. 22, 2021
(86) PCT No.: PCT/US2021/018967

§ 371 Date: Aug. 24, 2022

Related U.S. Application Data
(60) Provisional application No. 62/980,538, filed on Feb. 24, 2020.

(51) Int. Cl. A61K 41/00; C12N 5/078; C12N 13/00; A61K 35/19

(52) U.S. Cl. CPC A61K 41/0028; C12N 5/0644; C12N 13/00; A61K 35/19

(57) ABSTRACT

Methods are provided that utilize irradiation to selectively induce release of granules (such as granules containing cytokines or other therapeutically active substances) from platelets. Such irradiation can be performed ex vivo or in vivo, and can be utilized to provide a treated platelet suspension for therapeutic infusion.

This application claims the benefit of U.S. Provisional Patent Application No. 62/980,538 filed on Feb. 24, 2020. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling

### FIELD OF THE INVENTION

The field of the invention is activation of platelets.

### BACKGROUND

The following description includes information that may be useful in understanding the present invention. It is not an admission that any of the information provided herein is prior art or relevant to the presently claimed invention, or that any publication specifically or implicitly referenced is prior art.

Platelets are recognized as the primary cell regulating hemostasis and thrombosis, and are involved in inflammatory and immune responses during infection or injury. See Seong-Hoon Yun, et al., Platelet Activation: The Mechanisms and Potential Biomarkers, BioMed Research International, vol. 2016, Article ID 9060143, 5 pages, 2016. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Platelets can be activated by thrombin through protease-activated receptors (PAR) on the platelet surface through G protein-coupled receptors (GPCR). Activated platelets secrete several inflammatory mediators, including various proteins, chemokines, cytokines, and growth factors. Platelet-Rich Plasma (PRP) has been used to favor angiogenesis processes, promote proliferation of undifferentiated stem cells, and accelerate bone formation. See Jaehoon Choi et al., Archives of Plastic Surgery 2012;39(6):585-592.

Platelet granules are unique among secretory vesicles in both their content and their life cycle. Platelets contain three major granule types—dense granules, α-granules, and lysosomes (i.e., γ granules)—although other granule types have been reported (e.g., λ granules—contents involved in resorption during later stages of vessel repair.) Sharda A, Flaumenhaft R. The life cycle of platelet granules. F1000Res. 2018;7:236. Published 2018 Feb. 28. doi: 10.12688/f1000research.13283.1.

Previous work has used light irradiation on platelets. United States Patent Application Publication No. US 2017/0246470 A1 (Systems And Methods For Enhancing Platelet Biogenesis And Extending Platelet Lifespan With Low Level Light) by Meixiong Wu et al used low level light (LLL) to facilitate platelet biogenesis or extend platelet lifespan. United States Patent Application Publication No. US2010/0196497A1—Method of Treating Tissue Using Platelet-Rich Plasma in Combination with Low-Level Laser Therapy, by Lim et al, used platelet-rich plasma and laser energy (about 400 nm to 1500 nm, 1 mW to 500 mW, about 1 second to about 27.8 hours) to treat a patient's injured tissue.

Gre\check{ }ner et al. used green laser light irradiation on whole blood platelets and observed increased platelet cyclic GMP. The Effect Of Green Laser Light Irradiation On Whole Blood Platelets. Journal of Photochemistry and Photobiology B: Biology 79 (2005) 43-50. Prodouz K et al used visible light (450-600 nm) and a photosensitizer that resulted in a spontaneous release of serotonin, spontaneous aggregation, and marked morphological changes in platelets. Effects Of Two Viral Inactivation Methods On Platelets: Laser-UV Radiation And Merocyanine 540-Mediated Photoinactivation. Blood Cells. 1992;18(1):101-14; discussion 114-6. Monika Olban et al used low power (1-5 J) red laser at 670 nm to trigger the release of substances stored in the specific granules. The Biostimulatory Effect Of Red Laser Irradiation On Pig Blood Platelet Function. Cell Biology International Volume 22, Issue 3, March 1998, Pages 245-248.

However, prior work does not teach using specific frequencies to selectively trigger the release of contents from certain type of granules (e.g., α-granules), but not from other types (e.g., dense granules). Moreover, prior work does not teach using irradiation having wavelengths lower than 400 nm or higher than 1500 nm to trigger the release of contents from certain granules.

Thus, there is still a need for new methods of irradiating platelets to trigger the release of contents in specific granules.

### SUMMARY OF THE INVENTION

The inventive subject matter provides apparatus, systems and methods that utilize irradiation to induce release of granules (such as granules containing cytokines or other therapeutically active substances) from platelets.

One embodiment of the inventive concept is a method of treating a patient using an ex vivo method of triggering release of a cytokine or other therapeutic substance from a granule in a platelet, comprising by preparing a platelet rich solution containing platelets in suspension and irradiating the platelets to trigger release of the cytokine or other therapeutic substance into the platelet rich solution. The concentration of the cytokine so released is monitored, and the platelet rich solution is intravenously infused into a patient when concentration of the cytokine or other therapeutic substance in the platelet rich solution reaches a predetermined level. Suitable wavelengths for irradiation can range from about 10 nm to about 10,000 nm. For example, the irradiating energy can have a wavelength of from 1 nm to 10 nm, 10 nm to 100 nm, 100 nm to 200 nm, 200 nm 300 nm, 300 nm to 380 nm, 380 nm to 450 nm, 450 nm to 485 nm, 485 nm to 500 nm, 500 nm to 565 nm, 565 nm to 590 nm, 590 nm to 625 nm, 625 nm to 740 nm, 740 nm to 800 nm, 800 nm to 900 nm, 900 nm to 1,000 nm, 1,000 nm 2,000 nm, 2,000 nm to 3,000 nm, 3,000 nm to 4,000 nm, 4,000 nm to 5,000 nm, 5,000 nm to 6,000 nm, 6,000 nm to 7,000 nm, 7,000 to 8,000 nm, 8,000 nm to 9,000 nm, or 9,000 nm to 10,000 nm.

Another embodiment of the inventive concept is a method of treating a patient using an in vivo method of triggering release of a cytokine from a granule in a platelet, comprising by irradiating the patient (or a portion thereof) containing platelets to trigger release of the cytokine or other therapeutic substance. In preferred embodiments a highly vascular portion of the patient (such as a mucous membrane, oral membrane, nasal membrane, ear drum, eye, etc.) is irradiated. Suitable wavelengths for irradiation can range from about 10 nm to about 10,000 nm. For example, the irradiating energy can have a wavelength of from 1 nm to 10 nm, 10 nm to 100 nm, 100 nm to 200 nm, 200 nm 300 nm, 300 nm to 380 nm, 380 nm to 450 nm, 450 nm to 485 nm, 485 nm to 500 nm, 500 nm to 565 nm, 565 nm to 590 nm, 590 nm to 625 nm, 625 nm to 740 nm, 740 nm to 800 nm, 800 nm to 900 nm, 900 nm to 1,000 nm, 1,000 nm 2,000 nm, 2,000 nm to 3,000 nm, 3,000 nm to 4,000 nm, 4,000 nm to 5,000 nm, 5,000 nm to 6,000 nm, 6,000 nm to 7,000 nm, 7,000 to 8,000 nm, 8,000 nm to 9,000 nm, or 9,000 nm to 10,000 nm.

Another embodiment of the inventive concept is a method of triggering release of a cytokine or other therapeutic substance from a granule in a platelet, by determining a wavelength or wavelength that triggers release of the cytokine or other therapeutic substance from the platelet. A suspension of platelets is then exposed to the wavelength or wavelengths. Suitable wavelengths for irradiation can range from about 10 nm to about 10,000 nm. For example, the irradiating energy can have a wavelength of from 1 nm to 10 nm, 10 nm to 100 nm, 100 nm to 200 nm, 200 nm 300 nm, 300 nm to 380 nm, 380 nm to 450 nm, 450 nm to 485 nm, 485 nm to 500 nm, 500 nm to 565 nm, 565 nm to 590 nm, 590 nm to 625 nm, 625 nm to 740 nm, 740 nm to 800 nm, 800 nm to 900 nm, 900 nm to 1,000 nm, 1,000 nm 2,000 nm, 2,000 nm to 3,000 nm, 3,000 nm to 4,000 nm, 4,000 nm to 5,000 nm, 5,000 nm to 6,000 nm, 6,000 nm to 7,000 nm, 7,000 to 8,000 nm, 8,000 nm to 9,000 nm, or 9,000 nm to 10,000 nm.

In some embodiments the irradiating energy can be polychromatic, incorporating two or more wavelengths or ranges of wavelengths. In such embodiments the irradiating energy can alternate between the two or more wavelengths or ranges of wavelengths during irradiation of the platelets.

The substance released upon irradiation can be a cytokine, P-selectin, platelet factor 4, transforming growth factor-β1, platelet-derived growth factor, fibronectin, B-thromboglobulin, Von Willebrand factor, fibrinogen, coagulation factor V, coagulation factor XIII, ADP, ATP, calcium, and/or serotonin. In some embodiments two or more such substances can be released upon irradiation.

In some embodiments, irradiation results in release or activation of granules within the platelets. In such embodiments the granule can represent a subset or subtype of the range of granules found within the platelets. For example, the granule can be an α granule, a dense granule, a subtype of dense granule a gamma granule, or a lambda granule.

Various objects, features, aspects and advantages of the inventive subject matter will become more apparent from the following detailed description of preferred embodiments, along with the accompanying drawing figures.

### DETAILED DESCRIPTION

The inventive subject matter provides apparatus, systems and methods in which irradiation energy of specific frequency is used to selectively trigger the release of contents from specific platelet granules. Different types of irradiation energy contemplated herein include visible lights, laser, heat, infra-red, electric field, plasma radiation, acoustic waves (including ultrasound), and an electromagnetic field (e.g., nuclear magnetic resonance (NMR)).

Various objects, features, aspects and advantages of the inventive subject matter will become more apparent from the following detailed description of preferred embodiments, along with the accompanying drawing figures in which like numerals represent like components.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

A platelet rich solution can be prepared according to Rachita Dhurat and M S Sukesh. Principles and Methods of Preparation of Platelet-Rich Plasma: A Review and Author's Perspective. J Cutan Aesthet Surg. 2014 October-December; 7(4): 189-197. Concentrated platelets can be prepared using the PRP (platelet-rich plasma) method:

1. Obtain whole blood by venipuncture in acid citrate dextrose (ACD) tubes
2. Do not chill the blood at any time before or during platelet separation.
3. Centrifuge the blood using a ‘soft’ spin.
4. Transfer the supernatant plasma containing platelets into another sterile tube (without anticoagulant).
5. Centrifuge tube at a higher speed (a hard spin) to obtain a platelet concentrate.
6. The lower ⅓rd is PRP and upper ⅔rd is platelet-poor plasma (PPP). At the bottom of the tube, platelet pellets are formed.
7. Remove PPP and suspend the platelet pellets in a minimum quantity of plasma (2-4 mL) by gently shaking the tube.

Alternatively, a platelet rich solution can be prepared using the Buffy coat method:

1. Whole blood should be stored at 20° C. to 24° C. before centrifugation.
2. Centrifuge whole blood at a ‘high’ speed.
3. Three layers are formed because of its density: The bottom layer consisting of RBCs, the middle layer consisting of platelets and WBCs and the top PPP layer.
4. Remove supernatant plasma from the top of the container.
5. Transfer the buffy-coat layer to another sterile tube.
6. Centrifuge at low speed to separate WBCs or use leucocyte filtration filter.

In preferred embodiments, a platelet rich solution is irradiated (i.e., subjected to irradiation, radiant energy, or a field of irradiation/radiant energy), in a manner sufficient to trigger the platelets to selectively release desired cytokine(s), growth factors, clotting factors, and other substances from a specific type of granule. This selective release can be triggered by control of the wavelength(s) of the irradiation as well as the time and/or intensity of exposure. As used herein with respect to release of contents from granules, the term “selectively release” means release of contents from certain types or subtypes of granules, but not from other types or subtypes of granules. The specific types of granules from which contents can be selectively released include (but are not limited to) α granules, dense granules, gamma granules, and lambda granules.

In some embodiments, contents from only one type of granule is released in response to irradiation or other energy exposure. For example, it is contemplated that contents from α-granules (e.g., fibrinogen) could be released in response to irradiation, while contents from dense granules (e.g., serotonin) would not be released. In another example, contents from dense granules (e.g., serotonin) could be released in response to irradiation, while contents from these α-granules (e.g., P-selectin) would not be released.

Also, in preferred embodiments, a platelet rich solution is subjected to a field of irradiation energy, in a manner sufficient to trigger the platelets to selectively release desired cytokine from a specific subtype of granule. Studies by Battinelli et al. and Chatterjee et al. indicate that different subtypes of granules, for example, α-granule subtypes, contain different granule cargos that are released in response to different agonists. See Sorting Out Platelet . . . -Granules Robert Flaumenhaft, MD, PhD, November-December 2011, Volume 8, Issue 6, the Hematologist https://www.hematology.org/Thehematologist/Diffusion/1217.aspx. Battinelli E M, Markens B A, Italiano J E Jr. Release of angiogenesis regulatory proteins from platelet alpha granules: modulation of physiologic and pathologic angiogenesis. Blood. 2011;118:1359-1369. Chatterjee M, Huang Z, Zhang W, et al. Distinct platelet packaging, release, and surface expression of proangiogenic and antiangiogenic factors on different platelet stimuli. Blood. 2011;117:3907-3911. It is contemplated that subtypes of α-granule may contain one or more of the following substances that have therapeutic application: P-selectin, platelet factor 4, transforming growth factor-β1, platelet-derived growth factor, fibronectin, B-thromboglobulin, Von Willebrand factor, fibrinogen, coagulation factor V, coagulation factor XIII. Subtypes of dense granule may contain one or more of the desired molecules: ADP, ATP, calcium, and serotonin.

Radiant energy utilized in methods of the inventive concept includes electromagnetic radiation having wavelengths ranging from 10 nm to 10,00 nm, and can be generated by any suitable source. Such electromagnetic radiation can be coherent or non-coherent, and can be polarized or have random polarization. In some embodiments the method can utilize two or more wavelengths or wavelength ranges. In such embodiments, the two or more wavelength ranges can be continuous or discontinuous. Similarly, methods of the inventive concept can employ a single source of electromagnetic radiation or two or more sources of electromagnetic radiation. In some embodiments, the irradiation energy has a wavelength of from 1 to 10 nm, 10 to 100 nm, 100 to 200 nm, 200 to 300 nm, 300 to 380 nm, 380 to 450 nm, 450 to 485 nm, 485 to 500 nm, 500 to 565 nm, 565 to 590 nm, 590 to 625 nm, 625 to 740 nm, 740 to 800 nm, 800 to 900 nm, 900 to 1,000 nm, 1,000 to 2,000 nm, 2,000 to 3,000 nm, 3,000 to 4,000 nm, 4,000 to 5,000 nm, 5,000 to 6,000 nm, 6,000 to 7,000 nm, 7,000 to 8,000 nm, 8,000 to 9,000 nm, and/or 9,000 to 10,000 nm.

It is contemplated that a combination of different irradiation energies can be used to irradiate platelets, including combinations of wavelengths. In some embodiments, the irradiation energy comprises two or more frequency ranges. The irradiation energy field can also have an intensity gradient. A preferred irradiation energy is a laser beam. In especially preferred embodiments, the laser beam is phase conjugated.

In methods of the inventive concept, platelets or a platelet-containing suspension can be treated with radiant energy for a predefined length of time. For example, platelets and/or a platelet containing suspension can be treated with radiant energy for a period of time ranging from 10 μsec to 48 hours (preferably for less than one hour). In some embodiments the platelets and/or platelet-containing suspension are treated in a single exposure. In other embodiment, the electromagnetic radiation can be provided as two or more pulses. Such pulses can be of identical or different lengths of time.

In some embodiments, activating platelets using irradiation energy is performed ex vivo. In preferred methods a fluid (e.g., blood) that contains the platelets to be activated is obtained from a patient (e.g., via venipuncture). The platelets to be activated are then separated from other cells present in the fluid (e.g., red cells and white cells). Separation can be achieved as noted above or by any suitable means, including centrifugation, flow cytometry, filtering, etc. The isolated platelets are then subjected to the irradiation (i.e., radiant) energy. The platelets can be subjected to the irradiation energy for less than one hour, for example for between 15 and 45 minutes. The release of cytokine or other substance with clinical utility into the solution in which the platelets are suspended can be monitored, for example with a chemical sensor. If necessary, the irradiation step can be repeated until a desired concentration of the cytokine or other substance with clinical utility is reached. After the released cytokine or other substance with clinical utility reaches a predetermined level, activated platelets and the released cytokine or other substance with clinical utility are reintroduced to the patient by, for example, intravenous injection. Alternatively, they can be injected directly to a specific site of the body where they are needed.

In other embodiments, activation of platelets using irradiation energy is achieved in vivo. In currently preferred embodiments, a portion of skin surface of the patient is subjected to the irradiation energy. In especially preferred embodiments, an area of the patient having a rich blood supply and/or minimal pigment in the skin is subjected to the irradiation energy. Contemplated areas include a mucous membrane, tympanic membrane, nasal cavity, mouth, throat, and/or eye of the patient. In such embodiments time of exposure to the irradiation energy can be used to modulate the amount of cytokine or other substance with clinical utility from the patient's platelets.

Inventors believe that such activated platelets can be used to treat a variety of conditions, including inflammatory conditions (e.g., arthritis, tendinitis), neurodegenerative conditions, cardiovascular disease, and joint and/or tendon injuries.

It should be apparent to those skilled in the art that many more modifications besides those already described are possible without departing from the inventive concepts herein. The inventive subject matter, therefore, is not to be restricted except in the spirit of the appended claims. Moreover, in' interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1-99. (canceled)

100. A method of treating a patient via an ex vivo method for triggering release of a substance from a granule in a platelet, comprising:
preparing a platelet rich solution containing the platelet in suspension;
subjecting the platelet to a field of irradiation energy, in a manner sufficient to trigger release of the cytokine into the platelet rich solution;
monitoring concentration of the substance in the platelet rich solution; and
intravenously infusing the platelet rich solution into the patient when concentration of the substance in the platelet rich solution reaches a predetermined level.

101. The method of claim 100, wherein the irradiation energy comprises two frequency ranges.

102. The method of claim 100, wherein the irradiation energy alternates between two frequency ranges.

103. The method of claim 100, wherein the substance is selected from the group consisting of a cytokine, P-selectin, platelet factor 4, transforming growth factor-β1, platelet-derived growth factor, fibronectin, B-thromboglobulin, Von Willebrand factor, fibrinogen, coagulation factor V, coagulation factor XIII, ADP, ATP, calcium, and serotonin.

104. The method of claim 100, wherein the granule is selected from the group consisting of α granule, dense granule, gamma granule, and lambda granules.

105. The method of claim 100, wherein the granule is an α granule.

106. The method of claim 100, wherein the granule is a dense granule.

107. A method of treating a patient via an in vivo method for triggering release of a substance from a granule in a platelet, comprising:
determining an irradiation energy frequency or range of frequencies suitable to trigger selective release of a substance from a platelet; and
exposing the patient to the irradiation energy, in a manner sufficient to trigger release of the cytokine platelets of the patient.

108. The method of claim 107, wherein the irradiation energy comprises two frequency ranges.

109. The method of claim 107, wherein the irradiation energy alternates between two frequency ranges.

110. The method of claim 107, wherein the substance is selected from the group consisting of a cytokine, P-selectin, platelet factor 4, transforming growth factor-β1, platelet-derived growth factor, fibronectin, B-thromboglobulin, Von Willebrand factor, fibrinogen, coagulation factor V, coagulation factor XIII, ADP, ATP, calcium, and serotonin.

111. The method of claim 107, wherein the granule is selected from the group consisting of α granule, dense granule, gamma granule, and lambda granules.

112. The method of claim 107, wherein the granule is an α granule.

113. The method of claim 107, wherein the granule is a dense granule.

114. A method of triggering release of a substance from a granule in a platelet, comprising:
preparing a platelet rich solution containing the platelet in suspension; and
subjecting the platelet to a field of irradiation energy, in a manner sufficient to trigger release of the cytokine into the platelet rich solution.

115. The method of claim 114, wherein the irradiation energy comprises two frequency ranges.

116. The method of claim 114, wherein the irradiation energy alternates between two frequency ranges.

117. The method of claim 114, wherein the substance is selected from the group consisting of a cytokine, P-selectin, platelet factor 4, transforming growth factor-β1, platelet-derived growth factor, fibronectin, B-thromboglobulin, Von Willebrand factor, fibrinogen, coagulation factor V, coagulation factor XIII, ADP, ATP, calcium, and serotonin.

118. The method of claim 114, wherein the granule is selected from the group consisting of α granule, dense granule, gamma granule, and lambda granules.

119. The method of claim 114, wherein the granule is an α granule or a dense granule.
